(19) United States
(12) Patent Application Publication
(10) Pub. No.: US 2024/0293028 A1
(43) Pub. Date: Sep. 5, 2024

(54) DETECTION OF CIRCULATING TUMOR CELLS USING TUMOR-TARGETED NIR AGENTS

(71) Applicant: On Target Laboratories, LLC, West Lafayette, IN (US)

(72) Inventor: Sumith A Kularatne, West Lafayette, IN (US)

(21) Appl. No.: 18/245,072

(22) PCT Filed: Sep. 16, 2021
(86) PCT No.: PCT/US2021/050596

§ 371 Date: Mar. 13, 2023

Related U.S. Application Data
(60) Provisional application No. 63/079,178, filed on Sep. 16, 2020.

(51) Int. Cl. A61B 5/00; A61B 5/1455; G01N 33/574

(52) U.S. Cl. CPC A61B 5/0071; A61B 5/14556; G01N 33/57492

(57) ABSTRACT

The present disclosure relates to methods and compositions for detecting circulating tumor cells (CTCs) using a compound comprising a targeting moiety and a fluorescence imaging agent.

### CROSS-REFERENCE TO RELATED APPLICATION

The present application claims priority to U.S. Provisional Patent Application No. 63/079,178, filed on Sep. 16, 2020, which is incorporated by reference herein in its entirety.

### BACKGROUND

The presence of pathogenic cells in body fluids, such as the bloodstream, or the spread of pathogenic cells from other sites to the bloodstream, is one of the essential factors that determines whether a diseased patient will survive. Blood-borne metastasis is initiated by cancer cells transported through the circulation from the primary tumor to vital distant organs, and is directly responsible for most cancer-related deaths. Addressing this challenge, however, is confounded by our limited understanding of the process by which tumor cells exit from their primary site, intravasate into the circulation, and establish distant lesions in the lung, brain, liver, or bone. A circulating tumor cell (CTC) is a cell that has shed into the vasculature or lymphatics from a primary tumor, and is carried around the body in the blood circulation. CTCs play a central role in tumor dissemination and metastases, which are ultimately responsible for most cancer deaths. With the development of novel systemic treatment, it may become more crucial to detect early occult metastatic spread. Technologies that allow for identification and enumeration of rare CTC from cancer patients' blood have already established CTC as an important clinical biomarker for cancer diagnosis and prognosis. Indeed, current efforts to robustly characterize CTC as well as the associated cells of the tumor microenvironment, such as circulating cancer-associated fibroblasts, are poised to unmask key insights into the metastatic process.

Published studies estimate that growing tumors shed anywhere from 10⁵ to 3×10⁶ CTCs/day/g malignant tissue. Thus, the measurement of CTC numbers in peripheral blood constitutes one of the most sensitive methods for assessing residual malignant disease. Indeed, recent clinical studies demonstrate that CTC analyses can predict outcomes in multiple cancers, including cancers of the breast, prostate, and colorectal tissues. Consequently, increased effort is being focused on improving methods for detecting CTCs in blood samples from cancer patients.

The detection of CTCs presents several advantages over traditional tissue biopsies. They are non-invasive, can be used repeatedly, and provide more useful information on metastatic risk, disease progression, and treatment effectiveness. Affinity-based methods take advantage of antigens that are differentially expressed by CTCs (positive enrichment, EpCAM is mostly used), or by blood cells (negative selection, e.g., CD45). Most commonly, magnetic beads are armed with antibodies for positive or negative separation. Columns or cartridges can also be used, and most recently, microchips have been coated with antibodies. Through this methodology, only a subset of the CTCs is captured from the patient sample, namely the EpCAM positive cells. However, as tumor cells exhibit heterogeneity and thus, there is a high variability of expression, resulting in some tumor cells having no or very low expression of EpCAM, they evade capture. Further, EpCAM being an epithelial cell biomarker limits the ability to capture CTC from epithelial tumors that show low or no EpCAM expression (e.g., renal cancer). The detection of non-epithelial tumors, such as melanomas and sarcomas, or CTCs that have undergone epithelial-to-mesenchymal (EMT) transition to form cancer stem cells (CSC) can be challenging. Technologies in this category also work through the reverse process of using CD45 antibody-based depletion of white blood cells to leave behind the CTC. Further hindrances are experienced when these platforms are microfluidic-based systems as they have limitations on the sample volumes it is able to process. The small volumes that are processed by microfluidic-based systems require extended processing times, such as the CTC-iChip, which can process 8 mL of whole blood/hour with and an additional 1 hour set-up time. Thus, only 8 mL can be processed over 2 hours. It is important to note that there is only one FDA approved platform on the market for CTC capture at this time, which is CellSearch®, a technology based on magnetic EpCAM Ab based separation.

Differences in cell density can also be used for enrichment. The best-known method based on density is Ficoll Hypaque separation, which separates red blood cells from nucleated cells and tumor cells remain with the nucleated cells. As an alternative property of tumor cells for enrichment, cell size is being used, based on the fact that tumor cells are larger than most blood cells. Agarwal et al. have developed a size-based microfilter for enrichment and detection of CTCs, which is highly efficient and faster than affinity-based separation techniques and can be used for a wide range of molecular applications for additional characterization of CTCs.

After enrichment, various technologies can be used to distinguish CTCs from the nonspecifically captured cells, including cytomorphological characterization of CTCs, immunohistochemical/immunofluorescent (IHC/IF) detection of tumor specific antigens, or various real-time polymerase chain reaction (RT-PCR) approaches. Immunocytochemical detection of CTC relies on antibody-based detection of cells using antibodies specific for epithelial cells. The most commonly used antibodies are cytokeratins. It is often combined with markers such as CD45 that identify the background blood (non-CTC) cells. Multiplex IHC/IF approaches enable simultaneous visualization of multiple markers on a single cell. Molecular characterization of CTCs is carried out by various strategies that include fluorescent in situ hybridization (FISH), comparative genomic hybridization (CGH), PCR-based techniques, RNA-seq, and immunofluorescence. These studies have shed light on the oncogenic profile and metastatic potential of CTCs, and have allowed the comparison of the genetic profile of tumor metastases and CTCs to that of their primary tumor counterpart.

In PCR methods, freshly isolated blood samples are screened for the presence of multiple cancer-specific transcripts. While highly sensitive, such PCR methods require considerable time and yield largely qualitative answers. Although more advanced PCR techniques such as real-time PCR can provide reasonably quantitative results, their interpretation can also be confused by nonspecific amplification of normal sequences closely related to cancer genes, and low-level expression of target cancer genes in noncancerous cells. In contrast, flow cytometry methods have the advantage of being faster, simpler to perform, and more quantitative, but they also suffer from problems associated with cancer specificity. Thus, most flow cytometry methods rely on antibodies that recognize not only malignant cells, but also some healthy cells that express malignant markers (e.g., epithelial cell antigens such as cytokeratin). Further, in cases where marker expression is weak or masked, the same assays can lead to false-negative results because of failure to detect masked malignant cells present in the sample. Therefore, a CTC detection method with the rapidity and case of flow cytometry, but the specificity and sensitivity of PCR could find utility in the clinic.

Current in vivo diagnostic imaging technologies such as computed tomography, MRI, and positron emission tomography can detect micro-metastases only to a resolution of ˜2-3 mm. To permit earlier detection of metastatic disease, an in vitro diagnostic test, based on magnetic bead sorting followed by immunostaining and fluorescence imaging, has recently been developed, and can detect ˜5 CTCs in 7.5 mL of human peripheral blood. Although this improvement in detection sensitivity will likely save lives, its usefulness is limited by the volume of blood that can be sampled, thereby compromising CTC detection during the initial phases of metastasis, in early stages of disease, or when CTC numbers are reduced by therapy. In contrast, intravital flow cytometry circumvents sampling limitations and renders quantitation of rare events (<1 CTC per ml) statistically significant by enabling analysis of the majority of a patient's blood volume (˜5 liters).

Intravital flow cytometry, which noninvasively counts rare CTCs in vivo as they flow through the peripheral vasculature. The method involves i.v. injection of a tumor-specific fluorescent agent followed by multiphoton fluorescence imaging or epi-fluorescence imaging or other technology to visualize superficial blood vessels to quantitate the flowing CTCs. Similarly, isolation of CTCs by affinity-based enrichment methods using flow cytometry or microscopic methods also need a tumor-specific fluorescent agent that selectively binds to CTCs.

To further increase signal-to-background ratios, a tumor-specific agent has to be selected that rapidly clears from circulation if left uncaptured by CTCs. A minimal amount of a fluorescent dye can be injected shortly before quantitation, and CTCs can be detected by invasive or non- invasive methods with the aid of a fluorescence endoscope, multiphoton fluorescence imaging, epi-fluorescence imaging, or camera. Since no bulky imaging instruments or radiation detectors are required, the spatiotemporal constraints on a medical practitioner activity are minimal. However, one of the inherent challenges in the fluorescent dye field is the development of tumor-specific and sensitive fluorescence agents. For most oncological applications, an ideal fluorescent contrast agent should: i) selectively accumulate in cancer cells, ii) clear rapidly from healthy tissues, iii) be visible at significant depths below a tissue surface, and iv) be nontoxic at clinically relevant concentrations. Motivated by the need for improved tumor specificity and sensitivity in CTCs, the inventors have looked for a biomarker that is solely expressed on cancer cells to deliver NIR dyes for use in CTCs. While several options were available to target biomarkers on CTCs, the inventors have elected to develop small molecule ligand-targeted fluorescent probes for CTC detection because of their superior pharmacokinetic (PK) and biologic properties. Although ligand targeted visible fluorescent agents (e.g., fluorescein, rhodamine B. DyLight 488, Alexa Flou 488, etc.) can be used to image and quantitate CTCs, those dyes have been ineffective as they do not penetrate deep tissue or the skin to detect rare CTCs in vivo as they flow through the peripheral vasculature. In addition, the excitation and emission spectra of visible range fluorescence dyes exhibit significant background noise such that the targeted CTCs may not easily be detected. Moreover, fluorescein-based dyes have disadvantages due to their low shelf-life stability, and a relatively high level of nonspecific background noise from collagen in the surrounding tissue. The absorption of visible light by biological chromophores, in particular hemoglobin, further limits the usefulness of dyes that incorporate fluorescein. This means that conventional dyes cannot readily detect CTCs in vivo as they flow through the peripheral vasculature that may be buried deeper than a few millimeters in the tissue. Furthermore, fluorescence from fluorescein is quenched at low pH (below pH 5). Therefore, near infrared (NIR) fluorescence dyes have many advantages over visible range fluorescence dyes. While Food and Drug Administration (FDA)-approved indocyanine green (ICG), a non-targeted NIR dye, has been used in certain oncological applications, it has significant limitations with respect to sensitivity and specificity for tumor identification, poor tumor-to-background ratio (TBR), and higher liver and GI tract uptake due to its non-targeted nature. In an effort to overcome drawbacks associated with CTC detection and quantitation, the inventors developed novel tumor-targeted, low molecular weight cyanine NIR dyes (i.e., folate-, PSMA-, CA IX-, Glut1-, FAP-, CCK2R-, etc. targeted NIR dyes). Each tumor-targeted NIR dye demonstrated a very high affinity and specificity for the requisite biomarker/receptor/protein that is overexpressed CTCs. Moreover, when standard NIR cyanine dyes are employed as a ligand-targeted fluorescent probe, no toxicity is generally observed, and the emitted fluorescence can often be detected in CTCs up to 2 cm beneath the tissue surface or skin.

CTCs provide unique opportunities for real-time monitoring of disease progression and treatment response. Development of increasingly more sensitive technologies, particularly EpCAM-independent approaches, as well as techniques for robust molecular and functional characterization of these cells, will offer clues to the mechanisms by which cancer develops resistance to therapies and spreads to distant organs. In parallel, the development of integrated culture and interrogation platforms for CTCs will be an exceptionally powerful oncology toolset for discovering new therapeutics and precision cancer management.

### SUMMARY

One aspect of the present technology is a method for detecting circulating tumor cells (CTCs) in a subject using a compound that comprises a targeting moiety and a fluorescence imaging agent, wherein the targeting moiety targets a receptor, antigen, or antibody. In some aspects, the method comprises contacting a bodily fluid of the subject with the compound for a time that allows for binding of the compound to at least one CTC of a target cell type, illuminating the CTCs with an excitation light of a wavelength that is absorbed by the compound, and detecting the optical signal emitted by the compound.

In some aspects, the subject is a mammal. In other aspects, the subject is a human. In some aspects, the subject has cancer. In another aspect, the cancer is early-stage cancer or metastatic cancer. In some aspects, the CTCs are shed from a tumor. In another aspect, the tumor is a primary tumor.

In some aspects, the detection of the CTCs is conducted ex vivo. In yet another aspect, the ex vivo detection is of CTCs in bodily fluids. In a further aspect, the bodily fluid is blood.

In some aspects, the detection of the CTCs is conducted in vivo. In a further aspect, this in vivo detection can be completed in real-time. In another aspect, the method is used to track and analyze the distribution and the phenotype of cancer cells. In a further aspect, the information is tracked through a software platform. In yet another aspect, the information tracked is delivered to a smartphone and/or smartwatch app.

In some aspects, the CTCs are further quantified after detection. In one aspect, flow cytometry is used to quantitate the CTCs.

One aspect of the present technology is a method for diagnosing a disease in a subject, wherein the method comprises the detection of CTCs in the subject using a compound that comprises a targeting moiety and a fluorescence imaging agent, wherein the targeting moiety targets a receptor, antigen, or antibody. In some aspects, the disease is cancer. In some aspects, the method comprises contacting a bodily fluid of the subject with the compound for a time and under conditions that allow for binding of the compound to at least one CTC, illuminating the CTCs with an excitation light of a wavelength that is absorbed by the compound, detecting the optical signal emitted by the compound, comparing the optical signal measured in the previous step with at least one control data set, wherein the at least one control data set comprises a fluorescent signal from the compound contacted with a biological sample that does not comprise CTCs, and diagnosing the subject based on the previous step.

One aspect of the present technology is a method for detecting CTCs to provide real-time monitoring, screening, and management of a subject having a disease, wherein the method comprises the detection of CTCs using a compound that comprises a targeting moiety and a fluorescence imaging agent, wherein the targeting moiety targets a receptor, antigen, or antibody. In some aspects, the method comprises contacting a bodily fluid of the subject with the compound for a time and under conditions that allow for binding of the compound to at least one CTC, illuminating the CTCs with an excitation light of a wavelength that is absorbed by the compound, and detecting the optical signal emitted by the compound. In some aspects, the disease is cancer. In a further aspect, the real-time monitoring, screening, and management is tracked through a software platform. In yet another aspect, the information tracked is delivered to a smartphone and/or smartwatch app.

One aspect of the present technology is a method of detecting the presence of CTCs to determine the likelihood of the recurrence or remission of a disease in a subject, wherein the method comprises the detection of CTCs using a compound that comprises a targeting moiety and a fluorescence imaging agent, wherein the targeting moiety targets a receptor, antigen, or antibody. In some aspects, the disease is cancer. In some aspects, the method comprises contacting a bodily fluid of the subject with the compound for a time and under conditions that allow for binding of the compound to at least one CTC, illuminating the CTCs with an excitation light of a wavelength that is absorbed by the compound, and detecting the optical signal emitted by the compound.

One aspect of the present technology is a method of detecting the presence of CTCs to determine the likelihood of response to surgical treatment, chemotherapy, immunotherapy, radiotherapy, hormonal therapy, wherein the method comprises the detection of CTCs using a compound that comprises a targeting moiety and a fluorescence imaging agent, wherein the targeting moiety targets a receptor, antigen, or antibody.

### BRIEF DESCRIPTION OF THE DRAWINGS

The above-mentioned and other features of this disclosure, and the manner of attaining them, will become more apparent, and the disclosure itself will be better understood by reference to the following description of embodiments of the disclosure taken in conjunction with the accompanying drawings.

FIGS. 1A-1J illustrate the determination of in vitro binding affinity and target specificity of tumor-targeted Near Infrared (NIR) fluorescent agents to human cancer cells.

FIG. 1A is an epifluorescence image of KB cells (folate receptor-positive (FRα⁺) human cervical cancer cell line).

FIG. 1B is an epifluorescence image of MDA-MB231 cells (FRα⁺human triple-negative breast cancer cell line).

FIG. 1C is an epifluorescence image of SKOV3 cells (FRα⁺human ovarian cancer cell line) with a folate-targeted NIR agent.

FIG. 1D is an epifluorescence image of 22Rv1 cells (PSMA⁺human prostate cancer cell line) with a PSMA-targeted NIR imaging agent.

FIG. 1E is an epifluorescence image of HEK cells (CCK2 receptor transfected human kidney cancer cell line) with a CCK2 receptor-targeted NIR imaging agent.

FIG. 1F is an epifluorescence image of SKRC52 cells (CA IX⁺human kidney cancer cell line) with a CA IX-targeted NIR imaging agent.

FIG. 1G is an epifluorescence image of A549 cells (FRα⁻human lung cancer cell line) with a folate-targeted NIR agent.

FIG. 1H is a deferential interference contrast (DIC) image of A549 cells (FRα⁻human lung cancer cell line) with a folate-targeted NIR agent

FIG. 1I is an epifluorescence image of PC3 cells (PSMA⁻human prostate cancer cell line) with a PSMA-targeted NIR imaging agent.

FIG. 1J is a deferential interference contrast (DIC) image of PC3 cells (PSMA⁻human prostate cancer cell line) with a PSMA-targeted NIR imaging agent.

FIGS. 2A-2C illustrate the determination of in vitro binding efficiency of a folate receptor-targeted NIR fluorescent agent to human cervical cancer cells.

FIG. 2A is an overlay of a fluorescence image with a Deferential Interference Contrast (DIC) image of cells by epi-microscopy.

FIG. 2B is the DIC image of FIG. 2A.

FIG. 2C is the epifluorescence image of FIG. 2A.

FIGS. 3A-3F illustrate the determination of interference of human blood to detect cancer cells using a folate-targeted NIR fluorescent agent.

FIGS. 3A and 3B are overlays of fluorescence images with DIC images of cells by epi microscopy.

FIGS. 3C and 3D are the DIC images of FIGS. 3A and 3B, respectively.

FIG. 3E and 3F are the epifluorescence images of FIGS. 3A and 3B, respectively.

FIGS. 4A-4F illustrate the determination of in vitro specificity of a folate-targeted NIR agent to label cancer cells in human blood.

FIGS. 4A and 4B illustrate the overlay of fluorescence images with DIC images of cells by epi microscopy.

FIGS. 4C and 4D are DIC images of FIGS. 4A and 4B, respectively.

FIGS. 4E and 4F are epifluorescence images of FIGS. 4A and 4B, respectively.

FIGS. 5A-5D illustrate control experiments showing the absence of nonspecific labeling of cells in human blood by a tumor-targeted NIR agent.

FIG. 5A is a DIC image and FIG. 5B is an epifluorescence image of human blood samples from a healthy subject without a tumor-targeted NIR agent.

FIG. 5C is a DIC image and FIG. 5D is an epifluorescence image of human blood samples incubated with a tumor-targeted NIR agent.

FIG. 6 is a screen capture of a video that was recorded using white-light imaging to count the number of circulating tumor cells (CTCs) passed a certain point at a certain time.

### DETAILED DESCRIPTION

It is to be understood that this invention is not limited to the particular methodology, protocols, cell lines, constructs, and reagents described herein and as such may vary. It is also to be understood that the terminology used herein is for the purpose of describing particular embodiments only and is not intended to limit the scope of the present invention, which will be limited only by the appended claims.

As used herein and in the appended claims, the singular forms “a”, “an”, and “the” include plural reference unless the context clearly indicates otherwise.

Unless defined otherwise, all technical and scientific terms used herein have the same meaning as commonly understood to one of ordinary skill in the art to which this invention belongs. Although any methods, devices, and materials similar or equivalent to those described herein can be used in the practice or testing of the invention, the preferred methods, devices, and materials are now described.

All publications and patents mentioned herein are incorporated herein by reference for the purpose of describing and disclosing, for example, the constructs and methodologies that are described in the publications, which might be used in connection with the presently described invention. The publications discussed herein are provided solely for their disclosure prior to the filing date of the present application. Nothing herein is to be construed as an admission that the inventors are not entitled to antedate such disclosure by virtue of prior invention or for any other reason.

The terms “functional group”, “active moiety”, “activating group”, “leaving group”, “reactive site”, “chemically reactive group”, and “chemically reactive moiety” are used in the art and herein to refer to distinct, definable portions or units of a molecule. The terms are somewhat synonymous in the chemical arts and are used herein to indicate the portions of molecules that perform some function or activity and are reactive with other molecules.

The term “amino acid” refers to naturally occurring and non-naturally occurring amino acids, as well as amino acid analogs and amino acid mimetics that function in a manner similar to the naturally occurring amino acids. Naturally encoded amino acids are the 20 common amino acids (alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine) and pyrrolysine and selenocysteine. Amino acid analogs refer to compounds that have the same basic chemical structure as a naturally occurring amino acid, i.e., an α carbon that is bound to a hydrogen, a carboxyl group, an amino group, and an R group, such as homoserine, norleucine, methionine sulfoxide, methionine methyl sulfonium. Such analogs have modified R groups (such as norleucine) or modified peptide backbones, but retain the same basic chemical structure as a naturally occurring amino acid.

Amino acids may be referred to herein by either their commonly known three letter symbols or by the one-letter symbols recommended by the IUPAC-IUB Biochemical Nomenclature Commission.

Aspects of the present technology generally relate to a method for detecting circulating tumor cells (CTCs) in a subject using a compound that comprises a targeting moiety and a fluorescence imaging agent, wherein the targeting moiety targets a receptor, antigen, or protein. The subject can be any mammalian subject, including, but not limited to a human subject. In some aspects, the compound is in the form a pharmaceutically acceptable salt. Pharmaceutically acceptable salts include, but are not limited to, sodium, potassium, ammonium, calcium, magnesium, lithium, cholinate, lysinium, and hydrogen salts. In some aspects, the compound is formulated as a composition. The composition may be pharmaceutically or therapeutically acceptable. In other aspects, the composition may comprise a pharmaceutically or therapeutically acceptable amount of the compound.

In some aspects, the CTCs are from a cancerous tumor, specifically a primary tumor. In some aspects, the cancer is selected from the group consisting of pancreatic, gastrointestinal, stomach, colon, ovarian, cervical, prostate, glioma, carcinoid, or thyroid, lung cancer, bladder cancer, liver cancer, kidney cancer, sarcoma, breast cancer, brain cancer, testicular cancer, or melanoma.

In certain aspects, the CTCs are characterized by an intact, viable nucleus. In other aspects, the CTCs lack EpCAM or cytokeratins, or are cytokeratin-positive and CD45-negative. In yet another aspect, the traditional CTCs are undergoing apoptosis (programmed cell death). In some methods, these apoptotic CTCs may be used for monitoring a response to treatment. In some aspects, this response can be monitored in real-time. In some aspects, the CTCs are in clusters, which are two or more individual CTCs bound together.

The targeting moiety of the compound targets a receptor, antigen, or protein. In some aspect, the targeting moiety can be used to detect CTCs that have folate receptors that bind to folic acid, a folic acid analog, or another folate receptor-binding molecule. In other aspects, the targeting moiety can be used to detect CTCs that have prostate-specific membrane antigen (PSMA) or another prostate cancer-specific binding molecule. In other aspects, the targeting moiety can be used to detect CTCs that have glutamate carboxypeptidase II, carbonic anhydrase IX (CA IX), fibroblast activation protein alpha, glucose transporter 1, cholecystokinin-2, or other receptors, antigens, and/or antibodies commonly found in cancer cells. In some aspects, the targeting moiety and fluorescence imaging agent may be joined by a linker or a spacer. The linker or spacer may be, for example, an amino acid or a peptide. Because not all cancers express the same receptor, antigen, and/or antibody, it is contemplated that several compounds that target different receptors, antigens, and/or antibodies can be used in series or in combination.

In one aspect of the method, a bodily fluid from the subject is contacted with the compound. The bodily fluid includes, but is not limited to, urine, nasal secretions, nasal washes, bronchial lavages, bronchial washes, spinal fluid, sputum, gastric secretions, reproductive tract secretions (e.g., seminal fluid), lymph fluid, mucus, and blood. In some aspects, the compound is in contact with the bodily fluid for at least 30 minutes, alternatively at least 1 hour, alternatively at least 2 hours, alternatively at least 3 hours.

After the compound contacts the bodily fluid for a time that allows for binding of the compound to at least one CTC, the CTC(s) are illuminated with an excitation light of a wavelength that is absorbed by the compound.

In some aspects, the imaging agent is detectable outside the visible light spectrum. In some aspects, the imaging agent is greater than the visible light spectrum. In some aspects, the imaging agent is a fluorescence imaging agent with an excitation and emission spectra in the near-infrared range. The fluorescence imaging agent may have an absorption and emission maxima between about 600 nm and about 1000 nm, alternatively between about 600 nm and about 850 nm, alternatively between about 650 nm and about 850 nm. In some aspects, the method comprises subjecting the compound to an excitation light source and detecting fluorescence from the compound. In some aspects, the excitation light source may be near-infrared wavelength light. In some aspects, the excitation light wavelength is within a range from about 600 to about 1000 nanometers. In some aspects, the excitation light wavelength is within a range from about 670 to about 850 nanometers.

Light having a wavelength range from 600 nm and 850 nm lies within the near-infrared range of the spectrum, in contrast to visible light, which lies within the range from about 400 nm to about 500 nm. The excitation light may be monochromatic or polychromatic. In this manner, the compounds of the present disclosure are advantageous as they eliminate the need for the use of filtering mechanisms that would be used to obtain a desired diagnostic image if the fluorescence imaging agent is one that fluoresces at wavelengths below about 600 nm.

In some aspects, the compound may have one or more fluorescence imaging agents; alternatively, two more fluorescence imaging agents, wherein each fluorescence imaging agent has a signal property that is distinguishable from the other. Those of skill in the art will be able to devise combinations of successively administered fluorescence imaging agents, each of which specifically binds to the target site. In some examples, it may be desirable to include fluorophores in targeting constructs targeted to normal cells and the compounds of the present disclosure targeted to CTCs such that the contrast between the CTCs and cells are further enhanced to aid the observer in determining the location and size of the CTCs. The use of such additional fluorophores and targeting agents, in addition to the compounds of the present disclosure, provides the advantage that any natural fluorescence emanating from normal cells are obscured by the fluorescence emanating from fluorophore(s) in supplemental targeting constructs targeted to the normal cells. The greater the difference in color between the fluorescence emanating from normal cells and target CTCs, the easier it is for the observer to visualize the outlines and size of the target CTCs. Those of skill in the art can readily select a combination of fluorophores that present a distinct visual color contrast.

The spectrum of light used in the practice of the disclosed method is selected to contain at least one wavelength that corresponds to the predominate excitation wavelength of the fluorescence imaging agent. In some aspects, the method employs laser-induced fluorescence, laser-stimulated fluorescence, or light-emitting diodes.

In one aspect, the optical signal emitted by the compound is detected. The means used to detect the compounds vary based on factors including the identity of the imaging agent, whether the method is being practiced in vitro, in vivo, or ex vivo, and when practiced in vivo, the location in the subject to be visualized. However, suitable detection methods include, but are not limited to, immunofluorescence and immunocytochemistry, FISH (fluorescence in situ hybridization), SE-iFISH (immunostaining-FISH combined with subtraction enrichment), and FACS (fluorescence assisted cell sorting). Some in vivo diagnostic imaging technologies such as computed tomography, MRI, and positron-emission tomography can detect micro-metastases to a resolution of 2-3 mm. In some aspects, to permit earlier detection of cancer, in particular metastatic cancer, an in vitro diagnostic method may be employed, which has at least a 1.5 increase in sensitivity over some in vivo methods. However, in vitro methods may be limited by the volume of bodily fluids required. Intravital, such as intravital flow cytometry, allows for the analysis of the majority of a subject's blood volume, circumvents sampling limitations, and renders quantitation of rare events (<1 CTC per ml) statistically significant. Ex vivo flow cytometry allows for quantitation of small blood (e.g., 2 mL or less) and allows for further characterization and sorting.

In one aspect of the present technology, the detection of the CTCs is conducted ex vivo. This allows for a non-invasive or minimally invasive collection of a subject's bodily fluid(s). The term “minimally invasive”, as used herein, employs techniques that limit the size of incisions needed and so lessen wound healing time, associated pain, and risk of infection, and can include surgery. The term “non-invasive”, as used herein, refers to procedures that do not require an incision and do not break the skin to reach an intervention site. The bodily fluid includes, but is not limited to, urine, nasal secretions, nasal washes, bronchial lavages, bronchial washes, spinal fluid, sputum, gastric secretions, reproductive tract secretions (e.g., seminal fluid), lymph fluid, mucus, and blood.

In some aspects of an ex vivo method, blood samples from a cancer patient are collected. The volume of blood collected can be at least 500 μL, alternatively at least 1 mL, alternatively at least 1.5 mL, alternatively at least 2 mL, alternatively at least 2.5 mL, alternatively at least 3 mL, alternatively at least 3.5 mL, alternatively at least 4 mL, alternatively at least 4.5 mL, alternatively at least 5 mL, alternatively at least 5.5 mL, alternatively at least 6 mL, alternatively at least 6.5 mL, alternatively at least 7 mL, alternatively at least 7.5 mL, alternatively at least 8 mL, alternatively at least 8.5 mL, alternatively at least 9 mL, alternatively at least 9.5 mL, or alternatively at least 10 mL. In this method, the CTCs may be enriched and/or isolated using magnetic beads, buffy coat isolation, or CTC enrichment methods known in the art that incorporate the compound of the present invention or a composition that comprises the compound. After enrichment, the CTCs may be isolated by a method known in the art including, but not limited to ficoll, size-based enrichment, rosettesep, magnetophoretic mobility-based separation, microfluidic devices, fast (fiber-optic array scanning technology), flow cytometry, confocal microscopy, two-photon microscopy, epifluorescence microscopic methods

In some aspects, the detection of the CTCs is conducted in vivo. An in vivo detection of CTCs is desirable if there are blood volume limitations with an ex vivo approach. In some aspects, a medical-grade wire or catheter is coated with a composition comprising a compound that comprises a targeting moiety and a fluorescence imaging agent, wherein the targeting moiety targets a receptor, antigen, or antibody. In other aspects, the compound or a composition comprising the compound is administered orally, sublingually, intranasally, intraocularly, rectally, transdermally, mucosally, pulmonary, topically, or parenterally administration. Parenteral modes of administration include without limitation, intradermal, subcutaneous (s.c., s.q., sub-Q, Hypo), intramuscular (i.m.), intravenous (i.v.), intraperitoneal (i.p.), intra-arterial, intramedullary, intracardiac, intra-articular (joint), intrasynovial (joint fluid area), intracranial, intraspinal, and intrathecal (spinal fluids).

During an in vivo procedure, the target CTCs bind to the receptor, antigen, or antibody on the targeting moiety. The bound CTCs are then illuminated at an excitation light of a wavelength that is absorbed by the compound, and the optical signal emitted by the compound is detected. In some aspects, the compound is in contact with the bodily fluid for at least 30 minutes, alternatively at least 1 hour, alternatively at least 2 hours, alternatively at least 3 hours.

The nature of in vivo detection is that it allows for real-time monitoring of CTCs. In some aspects, the method can be used to track and analyze the distribution and the phenotype of cancer cells. This real-time analysis may be tracked through a software platform so that a physician may actively monitor a subject's CTCs. Additionally, the software program may provide algorithms to assist in quantifying CTCs and diagnosing disease. The algorithms may also allow for the computation of CTC trajectory and speed. The information tracked may also be provided to a subject through a smartphone and/or smartwatch app. In some aspects, the smartphone or smartwatch may provide a notification if a certain value with respect to the CTC levels is outside a pre-defined range.

In some methods, the CTCs are further quantified after detection. The CTCs can be quantified using techniques and methods including, but not limited to, ficoll, size-based enrichment, rosettesep, magnetophoretic mobility-based separation, microfluidic devices, fast (fiber-optic array scanning technology), flow cytometry, confocal microscopy, two-photon microscopy, or epifluorescence microscopic methods. In some aspects, flow cytometry, particularly multiphoton flow cytometry, is employed to detect and/or to quantitate the pathogenic cells.

In some in vivo methods, a compound of the present invention or a composition comprising the compound is administered to a subject with cancer. After 1-2 hrs post-administration, CTCs can be detected using two-photon microscopy, epifluorescence microscopic, or an innovative wearable, including but not limited to, a smartwatch, a wrist band, earpiece, wearable microscope, or bicep band, that can detect the fluorescent signal.

In an exemplary embodiment, sensors and underlying algorithms are the basis for detecting and quantifying a subject's CTC levels. If an abnormal CTC level is detected, i.e., a level higher or lower than a predetermined range, the subject is notified of the potential abnormality. In addition to receiving the notification, the subject can access more information related to these abnormalities on a software platform or app. Within the software platform or app, the user can see information including, but not limited to, the times when the algorithm identified an abnormality and a record of current and past CTC levels. In some embodiments, the innovative wearable, software, and/or app may be provided to a subject who has received a medical-grade wire or catheter coated with a composition comprising a compound that comprises a targeting moiety and a fluorescence imaging agent, wherein the targeting moiety targets a receptor, antigen, or antibody.

In another exemplary embodiment, the innovative wearable is a wearable microscope. The wearable microscope can detect and monitor CTCs labeled with the compound of the present invention. In some embodiments, the wearable microscope employs lasers to generate a fluorescent image allowing for the continuous monitoring of CTC levels. An algorithm can then process the fluorescent image, said algorithm being the basis for detecting and quantifying a subject's CTC levels. If an abnormal CTC level is detected, i.e., a level higher or lower than a predetermined range, the subject is notified of the potential abnormality via the wearable microscope, software platform and/or app.

In some aspects, the present technology can be used in a method for diagnosing a disease in a subject. In this aspect, the method comprises the additional step of comparing the optical signal measured in a previous method step with at least one control data set, wherein the at least one control data set comprises a fluorescent signal from the compound contacted with a biological sample that does not comprise CTCs, and diagnosing the subject based on the previous step.

In some aspects, the present technology may be used in a method for detecting CTCs to provide real-time monitoring, screening, and management of a subject having a disease.

In some aspects, the present technology may be used in a method of detecting the presence of CTCs to determine the likelihood of the recurrence or remission of a disease in a subject.

In an exemplary embodiment, a compound that comprises a targeting moiety and a fluorescence imaging agent, wherein the targeting moiety targets a receptor, antigen, or antibody is administered to a human or animal subject. After 30 minutes, to allow for clearance of unbound compounds, blood is drawn from the subject, and multiphoton intravital microscopy is used to detect CTCs. To achieve a quantitative analysis of these CTCs in larger, faster flowing vessels, fluorescence scanning is reduced to a single dimension along a transect perpendicular to the vessel. This modification allows an increase in scan rate from 2 to 500 frames per second.

In an exemplary embodiment, CTCs originating from a primary solid tumor are quantitated in vivo before the metastatic disease is detectable by microscopic examination of necropsied tissues.

In an exemplary embodiment, CTCs from a human or animal subject with cancer are detected in whole blood. The human or animal subject is treated with a compound that comprises a targeting moiety and a fluorescence imaging agent, wherein the targeting moiety targets a receptor, antigen, or antibody, and the collected blood samples are examined by flow cytometry. To confirm the labeled CTCs are malignant, the peripheral blood samples from the subjects are labeled with a monoclonal antibody and an appropriate secondary antibody conjugated to a fluorescence imaging agent.

It will be apparent to those skilled in the art that various changes may be made in the disclosure without departing from the spirit and scope thereof. Therefore, the disclosure encompasses embodiments in addition to those specifically disclosed in the specification and as indicated in the appended claims.

The examples that follow are merely provided for the purpose of illustrating particular embodiments of the disclosure and are not intended to be limiting to the scope of the appended claims. As discussed herein, particular features of the disclosed compounds and methods can be modified in various ways that are not necessary to the operability or advantages they provide. For example, the compounds can incorporate a variety of amino acids and amino acid derivatives, as well as targeting ligands depending on the particular use for which the compound will be employed. One of skill in the art will appreciate that such modifications are encompassed within the scope of the appended claims.

### Example 1

Cell culture: KB cells (FRGα⁺human cervical cancer cell line that expresses), MDA-MB231cells (FRGα⁺human triple negative breast cancer cell line), SKOV3 cells (FRGα⁺human ovarian cancer cell line), 22Rv1 cells (PSMA⁺human prostate cancer cell line), HEK cells (CCK2 receptor transfected human kidney cancer cell line), SKRC52 cells (CA IX⁺human kidney cancer cell line), A549 cells (FRα⁻human lung cancer cell line), and PC3 cells (PSMA⁻human prostate cancer cell line) were obtained from ATCC (Rockville, MD), and grown as a monolayer using folate free or normal 1640 RPMI-1640 medium (Gibco, NY) containing 10% heat-inactivated fetal bovine serum (Atlanta Biological, GA) and 1% penicillin streptomycin (Gibco, NY) in a 5% carbon dioxide: 95% air-humidified atmosphere at 37° C. for at least six passages before they were used for the assays.

Fluorescence Microscopy: To determine in vitro binding affinity, cancer cells (20,000-50,000 cells/well in 1 mL) were seeded into poly-D-lysine microwell Petri dishes, and allowed to form monolayers over 12-24 h. Spent medium was replaced with fresh medium containing tumor-targeted NIR agent (100 nM), and cells were incubated for 45 min at 37° C. After rinsing with fresh medium (2×1.0 mL) and PBS (1×1.0 mL), fluorescence images were acquired using an epifluorescence microscopy.

To determine interference of human blood to detect cancer cells, cervical cancer cells (50,000 cells/well in 1 mL) were seeded into poly-D-lysine microwell Petri dishes, and allowed to form monolayers over 12 h. Spent medium was replaced with fresh medium containing tumor-targeted NIR agent (100 nM), and cells were incubated for 45 min at 37° C. After rinsing with fresh medium (2×1.0 mL) and PBS (1×1.0 mL), cells were resuspended in human blood (1.0 mL), and fluorescence images were acquired using an epifluorescence microscopy.

To determine in vitro specificity of tumor-targeted NIR agent to label human cancer cells in human blood, cervical cancer cells (50,000 cells/well in 1 mL) were seeded into poly-D-lysine microwell Petri dishes and allowed to form monolayers over 12 h. Spent medium was replaced with human blood containing tumor-targeted NIR agent (100 nM), and incubated for 45 min at 37° C. Fluorescence images were acquired using an epifluorescence microscopy without rinsing the blood.

Analysis of cancer cells labeled with tumor-targeted NIR agent: Human cervical cancer cells labeled with a folate receptor-targeted NIR agent were added to 500 mL of PBS, passed through a capillary tube with 1 mL/min flow rate, and video recorded using white-light imaging to count the number of circulating tumor cells (CTCs) passed certain point at certain time.

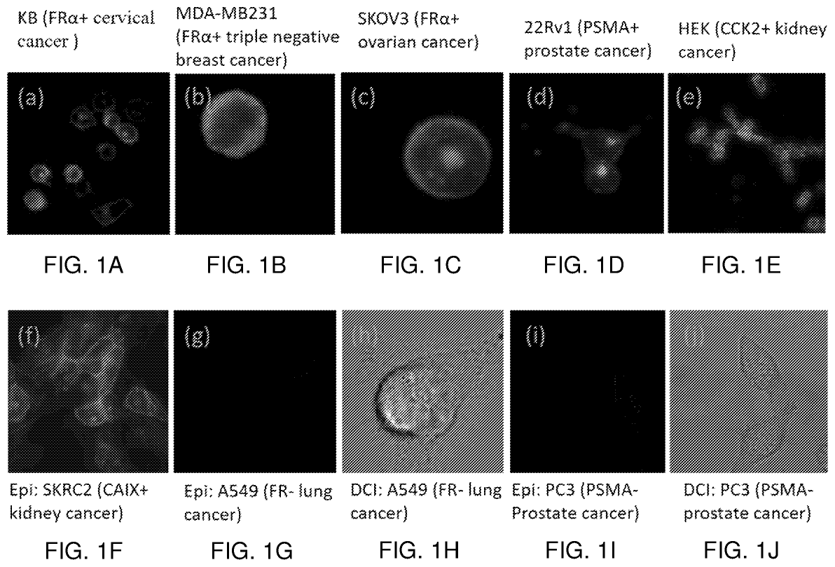

FIGS. 1A-1J are a determination of in vitro binding affinity and target specificity of tumor-targeted Near Infrared (NIR) fluorescent agents to human cancer cells. Human cancer cells were incubated with tumor-targeted NIR agents, washed with phosphate buffered saline (PBS) to remove unbound fluorescence agent, and imaged with epifluorescence (epi)-microscope. Epifluorescence images of (FIG. 1A) KB cells [folate receptor-positive (FRα⁺) human cervical cancer cell line), (FIG. 1B) MDA-MB231cells (FRGα⁺human triple negative breast cancer cell line), and (FIG. 1C) SKOV3 cells (FRGα⁺human ovarian cancer cell line) with folate-targeted NIR agent. Epifluorescence images of (FIG. 1D) 22Rv1 cells (PSMA⁺human prostate cancer cell line) with PSMA-targeted NIR imaging agent, (FIG. 1E) HEK cells (CCK2 receptor transfected human kidney cancer cell line) with CCK2 receptor-targeted NIR imaging agent, and (FIG. 1F) SKRC52 cells (CA IX⁺human kidney cancer cell line) with CA IX-targeted NIR imaging agent. Epifluorescence images of (FIG. 1G) A549 cells (FRα⁻human lung cancer cell line) with folate-targeted NIR agent and (FIG. 11) PC3 cells PSMA⁻human prostate cancer cell line) with PSMA-targeted NIR imaging agent. Deferential Interference Contrast (DIC) images of (FIG. 1H) A549 cells (FRα⁻human lung cancer cell line) with folate-targeted NIR agent and (FIG. 1J) PC3 cells (PSMA⁻human prostate cancer cell line) with PSMA-targeted NIR imaging agent.

Conclusion: These images show that tumor-targeted NIR agents efficiently label human cancer cells that expressed the biomarker (or receptor or targeted protein), but not the cancer cells that do not express the particular receptor/biomarker.

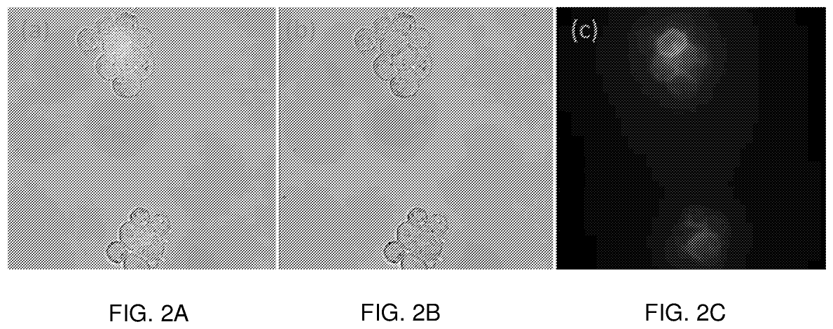

FIGS. 2A-2C are a determination of in vitro binding efficiency of folate receptor-targeted NIR fluorescent agent to human cervical cancer cells. Human cervical cancer cells were incubated with 100 nM of tumor-targeted NIR agent, washed with phosphate buffered saline (PBS) to remove unbound fluorescence agents, and imaged with epifluorescence (epi)-microscope. (FIG. 2A) overlay of fluorescence images with Deferential Interference Contrast (DIC) images of cells by epi-microscopy, (FIG. 2B) DIC images, and (FIG. 2C) epifluorescence images.

Conclusion: These images show that tumor-targeted NIR agent efficiently labels human cancer cells

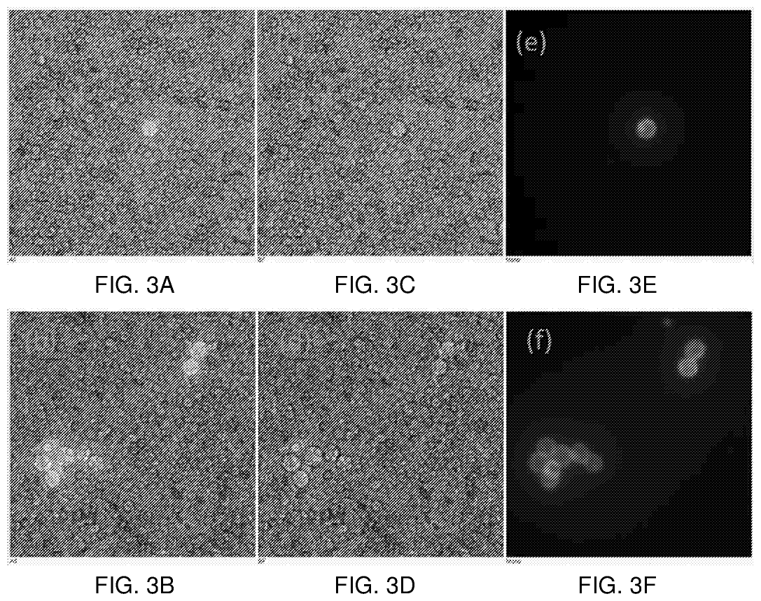

FIGS. 3A-3F are a determination of the interference of human blood to detect cancer cells using a folate-targeted NIR fluorescent agent. Human cancer cells were incubated with 100 nM of tumor-targeted NIR agent, washed with PBS to remove the unbound fluorescence agents, and the labeled cells added into human blood samples before imaging by epi-microscope. (FIGS. 3A and 3B) overlay of fluorescence images with DIC images of cells by epi microscopy. (FIGS. 3C and 3D) DIC images, and (FIGS. 3E and 3F) epifluorescence images.

Conclusion: These images show that there is no interference of human blood to detect cancer cells using a tumor-targeted NIR fluorescent agent.

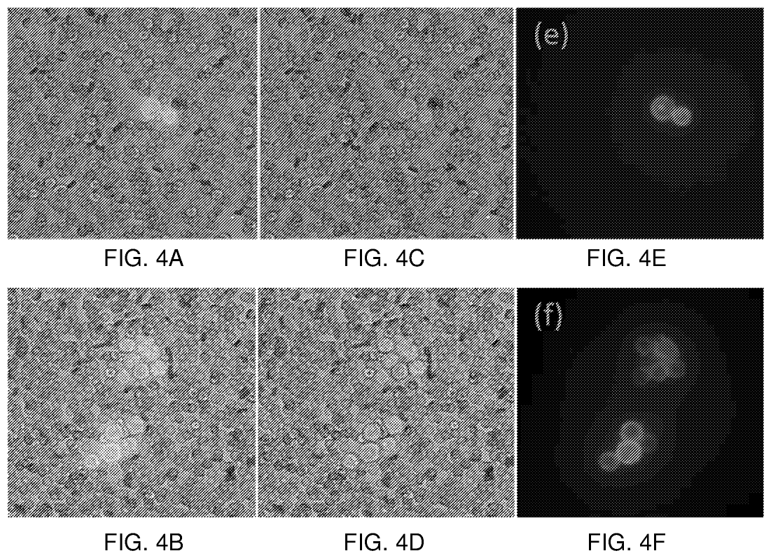

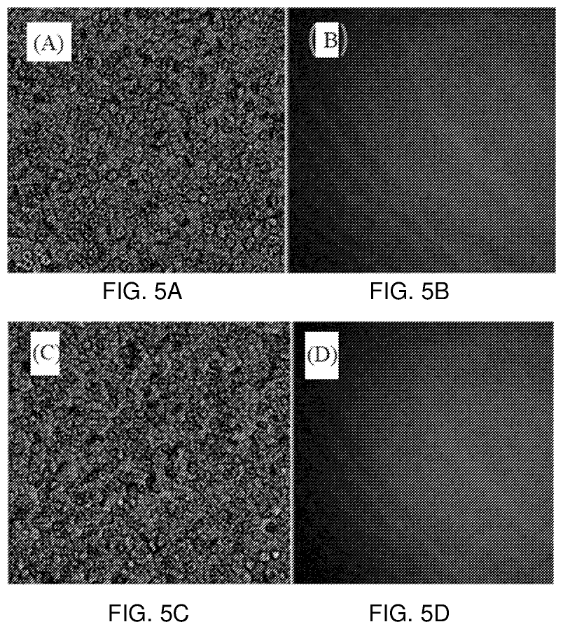

FIGS. 4A-4F are a determination of in vitro specificity of a folate-targeted NIR agent to label cancer cells in human blood. Human cancer cells in human blood were incubated with 100 nM of a tumor-targeted NIR agent and imaged with epi-microscope without isolating the cancer cells. (FIGS. 4A and 4B) overlay of fluorescence images with DIC images of cells by epi microscopy. (FIGS. 4C and 4D) DIC images, and (FIGS. 4E and 4F) epifluorescence images. FIGS. 5A-5D illustrate control experiments showing no nonspecific labeling of cells in the human blood by a tumor-targeted NIR agent. (FIG. 5A) DIC image and (FIG. 5B) epifluorescence image of human blood samples from a healthy subject without a tumor-targeted NIR agent; (FIG. 5C) DIC image and (FIG. 5D) epifluorescence image of human blood samples incubated with a tumor-targeted NIR agent.

Conclusion: These images show that tumor-targeted NIR agent selectively labeled the cancer cells without binding to any other cells in the blood samples. These images further indicate that human blood samples do not have any cells or protein fluorescence at NIR wavelength.

### Example 2

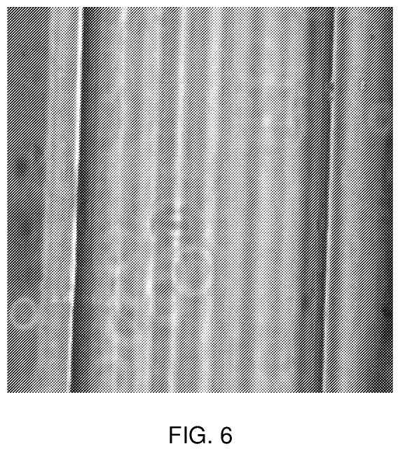

In an analysis of cancer cells labeled with a tumor-targeted NIR agent by phantom model, human cancer cells labeled with a tumor-targeted NIR agent were added to 500 mL of PBS, passed through a capillary tube with 1 mL/min flow rate, and video recorded using white-light imaging to count the number of circulating tumor cells (CTCs) passed a certain point at a certain time. (FIG. 6)

Conclusion: This demonstrates the ability to count CTCs after labeling the cells with a tumor-targeted NIR agent. This same phenomenon can be applied to cancer patients administered with a tumor-targeted NIR agent by monitoring the blood flow of a tissue such as a vein or a finger, wrist, bicep, ear, etc. with an NIR camera and digital counting apparatus.

Further aspects and embodiments of the present technology are described in the following paragraphs.

A method for detecting circulating tumor cells (CTCs) in a subject using a compound that comprises a targeting moiety and a fluorescence imaging agent, wherein the targeting moiety targets a receptor, antigen, or antibody.

A method for detecting CTCs to provide real-time monitoring, screening, and management of a subject having a disease, wherein the method comprises the detection of CTCs using a compound that comprises a targeting moiety and a fluorescence imaging agent, wherein the targeting moiety targets a receptor, antigen, or antibody.

A method of detecting the presence of CTCs to determine the likelihood of the recurrence or remission of a disease in a subject, wherein the method comprises the detection of CTCs using a compound that comprises a targeting moiety and a fluorescence imaging agent, wherein the targeting moiety targets a receptor, antigen, or antibody.

A method of detecting the presence of CTCs to determine the likelihood of response to surgical treatment, chemotherapy, immunotherapy, radiotherapy, hormonal therapy, wherein the method comprises the detection of CTCs using a compound that comprises a targeting moiety and a fluorescence imaging agent, wherein the targeting moiety targets a receptor, antigen, or antibody.

The methods described above wherein the method further comprises contacting a bodily fluid of the subject with the compound for a time that allows for binding of the compound to at least one CTC of a target cell type, illuminating the CTCs with an excitation light of a wavelength that is absorbed by the compound, and detecting the optical signal emitted by the compound.

A method for diagnosing a disease in a subject, wherein the method comprises the detection of CTCs in the subject using a compound that comprises a targeting moiety and a fluorescence imaging agent, wherein the targeting moiety targets a receptor, antigen, or antibody.

The method described above, wherein the method further comprises: contracting a bodily fluid of the subject with the compound for a time that allows for binding of the compound to at least one CTC, illuminating the CTCs with an excitation light of a wavelength that is absorbed by the compound, detecting the optical signal emitted by the compound, comparing the measured optical signal measured in the previous step with at least one control data set, wherein the at least one control data set comprises a fluorescent signal from the compound contacted with a biological sample that does not comprise CTCs, and diagnosing the subject based on the previous step.

The methods described above, wherein the subject is a mammal, alternatively a human. The methods described above, wherein the subject has a disease, alternatively cancer, alternatively early-stage cancer, or metastatic cancer.

The methods described above, wherein the cancer is selected from the group consisting of pancreatic, gastrointestinal, stomach, colon, ovarian, cervical, prostate, glioma, carcinoid, or thyroid, lung cancer, bladder cancer, liver cancer, kidney cancer, sarcoma, breast cancer, brain cancer, testicular cancer, and melanoma.

The methods described above, wherein the compound is a pharmaceutically acceptable salt, alternatively the pharmaceutically acceptable salt selected from the group consisting of sodium, potassium, ammonium, calcium, magnesium, lithium, cholinate, lysinium, and hydrogen salt.

The methods described above, wherein the compound is formulated as a composition, preferably wherein the composition comprises a pharmaceutically or therapeutically acceptable amount of the compound.

The methods described above, wherein the CTCs are from a cancerous tumor, specifically a primary tumor.

The methods described above, wherein the targeting moiety targets a folate receptor, Glutamate carboxypeptidase II, prostate-specific membrane antigen, carbonic anhydrase IX (CA IX), Fibroblast activation protein alpha, Glucose transporter 1, or cholecystokinin-2.

The methods described above, wherein the bodily fluid is selected from the group consisting of urine, nasal secretions, nasal washes, bronchial lavages, bronchial washes, spinal fluid, sputum, gastric secretions, reproductive tract secretions, lymph fluid, mucus, and blood.

The methods described above, wherein the compound is in contact with the bodily fluid for at least 30 minutes, alternatively at least 1 hour, alternatively at least 2 hours, alternatively at least 3 hours.

The methods described above, wherein fluorescence imaging agent has an excitation and emission spectra in the near-infrared range, alternatively the fluorescence imaging agent has an absorption and emission maxima between about 600 nm and 850 nm.

The methods described above, wherein the method is performed in vitro, in vivo, or ex vivo.

The methods described above, wherein the method is performed in vivo and CTCs are detected using two-photon microscopy, epifluorescence microscopic, or an innovative wearable.

The methods described above, wherein the innovative wearable is a smartwatch, a wrist band, earpiece, wearable microscope, or bicep band.

The methods described above, wherein the innovative wearable is a smartwatch, wherein the smartwatch employs sensors and algorithms for detecting and quantifying a subject's CTC levels.

The methods described above, wherein the innovative wearable is a wearable microscope, further wherein the wearable microscope employs lasers to generate a fluorescent image.

The methods described above, wherein if an abnormal CTC level is detected, the subject is notified of the potential abnormality.

The methods described above, wherein the CTC levels are continuously monitored.

The methods described above, wherein the method is used to track and analyze the distribution and the phenotype of cancer cells.

The methods described above, wherein the subject has cancer.

The methods described above, wherein the bodily fluid of the subject is contacted with the compound for at least 1 hour, alternatively at least 2 hours.

A method for detecting CTCs to provide real-time monitoring, screening, and management of subject having a disease, wherein the method comprises the detection of CTCs using a compound that comprises a targeting moiety and a fluorescence imaging agent, wherein the targeting moiety targets a receptor, antigen, or antibody and the real-time monitoring, screening, and management is tracked through a software platform or is delivered to an innovative wearable

The method described above, wherein the method comprises contacting a bodily fluid of the subject with the compound for a time and under conditions that allow for binding of the compound to at least one CTC, illuminating the CTCs with an excitation light of a wavelength that is absorbed by the compound emitted by the innovative wearable, and detecting the optical signal emitted by the compound.

The methods described above, wherein the detected CTCs are further isolated and/or enriched using ficoll, size-based enrichment, rosettesep, magnetophoretic mobility-based separation, microfluidic devices, fast (fiber-optic array scanning technology), flow cytometry, confocal microscopy, two-photon microscopy, or epifluorescence microscopic methods.

A medical-grade wire or catheter coated with a composition comprising a compound that comprises a targeting moiety and a fluorescence imaging agent, wherein the targeting moiety targets a receptor, antigen, or antibody, further wherein the targeting moiety targets a folate receptor, Glutamate carboxypeptidase II, prostate-specific membrane antigen, carbonic anhydrase IX (CA IX), Fibroblast activation protein alpha, Glucose transporter 1, or cholecystokinin-2, further wherein the fluorescence imaging agent has an excitation and emission spectra in the near-infrared range, further wherein the fluorescence imaging agent has an absorption and emission maxima between about 600 nm and 850 nm.

### References

Riquet, M; et al (2014). “Lymphatic spread of lung cancer: anatomical lymph node chains unchained in zones”. Revue de Pneumologie Clinique. 70 (1-2): 16-25.

He, Wei; et al (2008) “Quantitation of circulating tumor cells in blood samples from ovarian and prostate cancer patients using tumor-specific fluorescent ligands ” Int. J. Cancer: 123,1968-1973

Butler T P, Gullino P M.(1975) Quantitation of cell shedding into efferent blood of mammary adenocarcinoma. Cancer Res; 35:512-6.

Donelli M G, Rosso R, Garattini S. (1969) Quantitative studies on cancer dissemination. Cancer Res 29:414-18.

Liotta L A, Kleinerman J, Saidel G M. (1974) Quantitative relationships of intravascular tumor cells, tumor vessels, and pulmonary metastases following tumor implantation. Cancer Res 34:997-1004.

Koike A. (1964) Mechanism of blood-borne metastases. I. Some factors affecting lodgment and growth of tumor cells in the lungs. Cancer 17:450-60.

Cristofanilli M, Budd G T, Ellis M J, Stopeck A, Matera J, Miller M C, Reuben J M, Doyle G V, Allard W J, Terstappen L W, Hayes D F. (2004) Circulating tumor cells, disease progression, and survival in metastatic breast cancer. N Engl J Med 351:781-91.

Cristofanilli M, Hayes D F, Budd G T, Ellis M J, Stopeck A, Reuben J M, Doyle G V, Matera J, Allard W J, Miller M C, Fritsche H A, Hortobagyi G N, et al. (2005) Circulating tumor cells: a novel prognostic factor for newly diagnosed metastatic breast cancer. J Clin Oncol 2005;23: 1420-30.

Ghossein R A, Scher H I, Gerald W L, Kelly W K, Curley T, Amsterdam A, Zhang Z F, Rosai J. (1995) Detection of circulating tumor cells in patients with localized and metastatic prostatic carcinoma: clinical implications. J Clin Oncol 13:1195-200.

Sabbatini R, Federico M, Morselli M, Depenni R, Cagossi K, Luppi M, Torelli G, Silingardi V. (2000) Detection of circulating tumor cells by reverse transcriptase polymerase chain reaction of maspin in patients with breast cancer undergoing conventional-dose chemotherapy. J Clin Oncol 18:1914-20.

Seiden M V, Kantoff P W, Krithivas K, Propert K, Bryant M, Haltom E, Gaynes L, Kaplan I, Bubley G, DeWolf W. Detection of circulating tumor cells in men with localized prostate cancer. (1994) J Clin Oncol;12:2634-9.

Smith B M, Slade M J, English J, Graham H, Luchtenborg M, Sinnett H D, Cross N C, Coombes R C. (2000) Response of circulating tumor cells to systemic therapy in patients with metastatic breast cancer: comparison of quantitative polymerase chain reaction and immunocytochemical techniques. J Clin Oncol 18:1432-9.

Cohen S J, Alpaugh R K, Gross S, O'Hara S M, Smirnov D A, Terstappen L W, Allard W J, Bilbee M, Cheng J D, Hoffman J P, Lewis N L, Pellegrino A, et al. (2006) Isolation and characterization of circulating tumor cells in patients with metastatic colorectal cancer. Clin Colorectal Cancer;6:125-32.

Moreno J G, Miller M C, Gross S, Allard W J, Gomella L G, Terstappen L W. (2005) Circulating tumor cells predict survival in patients with metastatic prostate cancer. Urology;65:713-18.

Racila E, Euhus D, Weiss A J, Rao C, McConnell J, Terstappen L W, Uhr J W. Detection and characterization of carcinoma cells in the blood. (1998) Proc Natl Acad Sci USA;95:4589-94.

Krivacic R T, Ladanyi A, Curry D N, Hsieh H B, Kuhn P, Bergsrud D E, Kepros J F, Barbera T, Ho M Y, Chen L B, Lerner R A, Bruce R H. (2004) A rare-cell detector for cancer. Proc Natl Acad Sci USA;101: 10501-4.

Marrinucci, D; Bethel, K; Luttgen, M; Nieva, J; Kuhn, P; Kuhn, P (September 2009). “Circulating tumor cells from well-differentiated lung adenocarcinoma retain cytomorphologic features of primary tumor type”. Archives of Pathology & Laboratory Medicine. 133 (9): 1468-71.

Attard G, Swennenhuis J F, Olmos D, Reid A H, Vickers E, A'Hern R, Levink R, Coumans F, Moreira J, Riisnaes R, Oommen N B, Hawche G, Jameson C, Thompson E, Sipkema R, Carden CP, Parker C, Dearnaley D, Kaye S B, Cooper C S, Molina A, Cox M E, Terstappen L W, de Bono J S (2009). “Characterization of ERG, AR and PTEN gene status in circulating tumor cells from patients with castration-resistant prostate cancer”. Cancer Res. 69 (7): 2912-8.

Freedland S J, Partin A W. (2005) Detecting prostate cancer with molecular markers: uPM3. Rev Urol;7:236-8.

Lu K H, Patterson A P, Wang L, Marquez R T, Atkinson E N, Baggerly K A, Ramoth L R, Rosen D G, Liu J, Hellstrom I, Smith D, Hartmann L, et al. (2004) Selection of potential markers for epithelial ovarian cancer with gene expression arrays and recursive descent partition analysis. Clin Cancer Res 10:3291-300.

Lambrechts A C, van' t Veer L J, Rodenhuis S. (1998) The detection of minimal numbers of contaminating epithelial tumor cells in blood or bone marrow: use, limitations and future of RNA-based methods. Ann Oncol 9:1269-76.

Jung R, Petersen K, Kruger W, Wolf M, Wagener C, Zander A, Neumaier M. Detection of micrometastasis by cytokeratin 20 RT-PCR is limited due to stable background transcription in granulocytes. (1999) Br J Cancer;81:870-3.

Kruger W H, Jung R, Detlefsen B, Mumme S, Badbaran A, Brandner J, Renges H, Kroger N, Zander AR. (2001) Interference of cytokeratin-20 and mammaglobin-reverse-transcriptase polymerase chain assays designed for the detection of disseminated cancer cells. Med Onco1;18:33-8.

Chelly J, Concordet J P, Kaplan J C, Kahn A. Illegitimate transcription: transcription of any gene in any cell type.(1989) Proc Natl Acad Sci USA;86:2617-21.

Chelly J, Kaplan J C, Maire P, Gautron S, Kahn A. (1988) Transcription of the dystrophin gene in human muscle and non-muscle tissue. Nature;333:858-60.

Humphries S, Windass J, Williamson R. Mouse globin gene expression in erythroid and non-erythroid tissues. (1976) Cell;7:267-77.

He W, Wang H, Hartmann L C, Cheng J X, Low P S. In vivo quantitation of rare circulating tumor cells by multiphoton intravital flow cytometry. Proc Natl Acad Sci U S A. 2007 Jul. 10;104(28): 11760-5.

Agarwal A, Balic M, El-Ashry D, Cote R J. Circulating Tumor Cells: Strategies for Capture, Analyses, and Propagation. Cancer J. 2018 Mar/Apr;24(2):70-77

While the present invention has been described with reference to certain aspects, it will be understood by those skilled in the art that various changes may be made and equivalents may be substituted without departing from the scope of the present invention. In addition, many modifications may be made to adapt a particular situation or material to the teachings of the present invention without departing from its scope. Therefore, it is intended that the present invention not be limited to the particular embodiment disclosed, but that the present invention will include all aspects falling within the scope of the appended claims.

1. A method for detecting circulating tumor cells (CTCs) in a subject using a compound, or a pharmaceutically acceptable salt of the compound, comprising a targeting moiety and a fluorescence imaging agent, wherein the targeting moiety is selected from the group consisting of folate receptor, Glutamate carboxypeptidase II, prostate-specific membrane antigen, carbonic anhydrase IX (CA IX), Fibroblast activation protein alpha, Glucose transporter 1, or cholecystokinin-2.

2. The method of claim 1, wherein for detecting CTCs to
(a) provides real-time monitoring, screening, and management of a subject having a disease
(b) is used to determine a likelihood of recurrence or remission of a disease in a subject, and/or
(c) is used to determine a likelihood of response to surgical treatment, chemotherapy, immunotherapy, radiotherapy, or hormonal therapy.

3. (canceled)

4. (canceled)

5. The method of claim 1, wherein the method further comprises:
(a) contacting a bodily fluid of the subject with the compound for a time that allows for binding of the compound to at least one CTC of a target cell type,
(a) illuminating the CTCs with an excitation light of a wavelength that is absorbed by the compound, and
(a) and detecting an optical signal emitted by the compound.

6. The method of claim 1, wherein the detecting CTSs is used for diagnosing a disease in a subject.

7. The method of claim 6, wherein the method further comprises:
(a) contacting a bodily fluid of the subject with the compound for a time that allows for binding of the compound to at least one CTC,
(b) illuminating the CTCs with an excitation light of a wavelength that is absorbed by the compound,
(c) detecting an optical signal emitted by the compound,
(d) comparing the optical signal measured in step (c) with at least one control data set, wherein the at least one control data set comprises a fluorescent signal from the compound contacted with a biological sample that does not comprise CTCs, and
(e) and diagnosing the subject based on step (d).

8. The method of claim 1, wherein the subject is a mammal or a human.

9. (canceled)

10. The method of claim 1, wherein the subject has cancer and the cancer is selected from the group consisting of pancreatic, gastrointestinal, stomach, colon, ovarian, cervical, prostate, glioma, carcinoid, or thyroid, lung cancer, bladder cancer, liver cancer, kidney cancer, sarcoma, breast cancer, brain cancer, testicular cancer, melanoma, early-stage cancer and metastatic cancer.

11-14. (canceled)

15. The method of claim 1, wherein the pharmaceutically acceptable salt selected from the group consisting of sodium, potassium, ammonium, calcium, magnesium, lithium, cholinate, lysinium, and hydrogen salt.

16. The method of claim 1, wherein the compound is formulated as a composition, wherein the composition comprises a pharmaceutically or therapeutically acceptable amount of the compound.

17. The method of claim 1, wherein the CTCs are from a cancerous tumor.

18-20. (canceled)

21. The method of claim 1, wherein fluorescence imaging agent has an excitation and emission spectra in the near-infrared range.

22. (canceled)

23. (canceled)

24. The method of claim 1, wherein the method is performed in vivo and CTCs are detected using two-photon microscopy, epifluorescence microscopic, or an innovative wearable.

25-28. (canceled)

29. The method of claim 24, wherein if an abnormal CTC level is detected, the subject is notified of the potential abnormality.

30. The method of claim 24, wherein the CTC levels are continuously monitored.

31. The method of claim 24, wherein the method is used to track and analyze the distribution and the phenotype of cancer cells.

32. (canceled)

33. (canceled)

34. The method of claim 1, wherein the detected CTCs are further isolated and/or enriched using ficoll, size-based enrichment, rosettesep, magnetophoretic mobility-based separation, microfluidic devices, fast (fiber-optic array scanning technology), flow cytometry, confocal microscopy, two-photon microscopy, or epifluorescence microscopic methods.

35. A method for detecting CTCs to provide real-time monitoring, screening, and management of subject having a disease, wherein the method comprises the detection of CTCs using a compound that comprises a targeting moiety and a fluorescence imaging agent, wherein the targeting moiety is selected from the group consisting of folate receptor, Glutamate carboxypeptidase II, prostate-specific membrane antigen, carbonic anhydrase IX (CA IX), Fibroblast activation protein alpha, Glucose transporter 1, or cholecystokinin-2 and the real-time monitoring, screening, and management is tracked through a software platform or is delivered to an innovative wearable

36. The method of claim 35, wherein the method comprises contacting a bodily fluid of the subject with the compound for a time and under conditions that allow for binding of the compound to at least one CTC, illuminating the CTCs with an excitation light of a wavelength that is absorbed by the compound emitted by the innovative wearable, and detecting the optical signal emitted by the compound.

37. A medical-grade wire or catheter coated with a composition comprising a compound, the compound comprising a targeting moiety selected from the group consisting of folate receptor, Glutamate carboxypeptidase II, prostate-specific membrane antigen, carbonic anhydrase IX (CA IX), Fibroblast activation protein alpha, Glucose transporter 1, or cholecystokinin-2 and a fluorescence imaging agent, wherein the targeting moiety targets a receptor, antigen, or antibody.

38-40. (
